(19) United States
(12) Patent Application Publication
(10) Pub. No.: US 2022/0355003 A1
(43) Pub. Date: Nov. 10, 2022

(54) BLOOD TESTING DEVICE

(71) Applicant: Hemovent GmbH, Aachen (DE)

(72) Inventors: Oliver Marseille, Aachen (DE); Christopher Franzen, Aachen (DE)

(21) Appl. No.: 17/623,865

(22) PCT Filed: Jul. 9, 2020
(86) PCT No.: PCT/EP2020/069377

§ 371 Date: Dec. 29, 2021

(30) Foreign Application Priority Data
Jul. 9, 2019 (LU) LU101301

(51) Int. Cl. A61M 1/14

(52) U.S. Cl. CPC A61M 1/154; A61M 2205/3306; A61M 2205/3375; A61M 2205/3334; A61M 2230/20; A61M 2230/50; A61M 2230/30

(57) ABSTRACT

The invention concerns a blood testing device for the direct connection to a throughflow unit for blood, in particular a throughflow unit of an extracorporeal blood circuit, and for the recording of blood parameters of the blood flowing through the throughflow unit. The blood testing device can be operated without cables.

The invention concerns a blood testing device which can be connected directly to a throughflow unit for blood, in particular a throughflow unit of an extracorporeal blood stream, and can record blood parameters of the blood flowing through the throughflow unit.

Furthermore, the invention concerns a throughflow unit which is designed and intended to be added in an extracorporeal blood stream so that blood flows along a flow path through the throughflow unit and which is designed to be coupled directly to a blood testing device according to the invention.

The invention also concerns a system which has a blood testing device of this type as well as a throughflow unit of this type.

It is the task of the present invention to provide an improved blood testing device.

The task is resolved by a blood testing device which is characterised in that the blood testing device can be operated without wires.

It is also the task of the present invention to provide a throughflow unit which enables, by means of a blood testing device which can be connected to it, different blood parameters to be simply and quickly determined immediately in a variety of ways.

The task is resolved by a throughflow unit which is characterised in that the throughflow unit has at least one attachable transducer element and at least one throughflow device transducer.

In particular, the blood testing device according to the invention can be designed to be self-sufficient. The blood testing device according to the invention is particularly advantageous in that it is fast and can be used without having to connect it to a power supply or to involve other devices. The blood testing device according to the invention is operated without cables so that, on the one hand, it is particularly easy to manage, and, on the other hand, it does not obstruct the personnel handling the patient with hanging cables and even avoids detaching the cables by the personnel circulating around. It is unnecessary to attach it to other devices for control, operation, power supply or display of the data by a visual display or by alarms. Thus, it can be used anywhere where the measurement of one or all parameters in a blood-carrying vessel needs to be determined. Possible places where it can be used are in the areas of heart-lung machines, ECMO and ECLS (support for artificial lungs, heart or heart circuits), organ perfusion and other organ substitutions or support systems.

The device can have a display for displaying the desired measurement parameters and one or more control knobs with which the display of the measurement parameters and alarms can be selected or changed. Also the device can be activated simply by the control knob as required in order to save power.

Furthermore, the blood testing device is preferably designed as a portable device and is compact, so that it can be carried by one person and is easy to use. Thus, the device can be lighter than 1 kg, in particular lighter than 0.5 kg and has a housing smaller than 10×10×10 cm, in particular less than 10×6×6 cm. In particular, the device is made so that it does not need a support but instead is simply attached to the blood carrying vessel.

In an advantageous embodiment, the blood testing device has a housing in or on which an electrical energy storage system and an indicator device to display the recorded blood parameters are arranged which can be supported or substituted by a cable, such as a USB cable, also charged during the operation of the internal current source. The optional connection can also be used to transmit or read out data. Charging by means of energy transmission is also possible.

In a particular embodiment, the blood testing device has a device for the wireless transmission of data, such as via Bluetooth or an RF link, for external monitors. If, after transportation, a patient enters a clinic environment, the blood testing device can be plugged directly into the clinic monitoring system.

In a particular embodiment, the blood testing device has a transducer. In particular, the transducer can be designed and specified such that, with a transducer connecting element, a throughflow unit can be operatively connected. The transducer connecting element can be a window or a membrane, for example, which is explained below in more detail. Basically. It is advantageously provided that an operative connection between the transducer of the blood testing device and a transducer connecting element of a throughflow unit is created to measure blood values. In order to create the operative connection, preferably a positive-locking connection is established between the blood testing device and the throughflow unit. Furthermore, the connection is preferably made such that the transducer fits close to the transducer connecting element or contacts it directly.

An embodiment is particularly advantageous where the transducer has at least one light source to illuminate the blood flowing through a throughflow unit. By so doing, a specific light transmission into the blood can take place by means of the transducer and, due to the shining of the light, detection light emitted from the blood (e.g. reflected light, scattered light or fluorescent light) can be detected by a light sensor of the transducer and, if necessary, be analysed in particular with regard to the light power and/or the wavelength.

Alternatively or in addition, provision can be made advantageously that the transducer has at least one sound source to apply sound to the blood flowing through a throughflow unit. Using this method, a certain sound input can be made in the blood by means of the transducer. A sound sensor can also be provided which reflects sound from the blood or receives scattered sound. A transducer of this type can be an ultrasound transducer in particular which works based on ultrasound and functions as a flow meter.

An embodiment is particularly advantageous in which the transducer has both a light source as well as a sound source. As a result, both blood parameters which are provided with transducers with a light source as well as with a sound source can be recorded simultaneously.

Simple manageability is provided by one embodiment in which the blood testing device according to the invention has a transducer interface which is designed and intended to be operatively connected to a throughflow transducer incorporated in a throughflow unit. This enables the blood testing device to be used together with a throughflow unit which contains an integrated transducer, which enables an uncomplicated and fast connection to be made, in particular without requiring additional cable connections.

In particular, the transducer interface can be designed advantageously such that power from an electrical energy storage unit of the blood testing device is transmitted to a throughflow transducer device incorporated in a throughflow unit and/or is designed to forward measurement signals from a throughflow device transducer incorporated in a throughflow unit. In this manner, this enables immediate deployment of the transducer throughflow device incorporated in the throughflow unit by connecting to the blood testing device according to the invention without having to connect additional cable connections to power or signal transmission facilities.

In particular, the transducer can be preferably a pressure transducer or a flow transducer or an optical transducer. In particular, it is also possible that several transducers, in particular, of different types, can be present. In this manner it is possible to achieve different technical investigation possibilities, in particular even in combination.

In a simply manageable embodiment of the blood testing device, a holder is present, in or on which a throughflow unit, in particular non-destructive and/or without tools, can be attached. The holder enables a fast and efficient, in particular non-destructive coupling or uncoupling of a throughflow unit. Furthermore, an immediately recognisable connection possibility by the holder is created for a user by means of which an operator error can be reduced due to erroneous coupling. In particular, this guarantees that the blood testing device and the throughflow direction can be coupled together correctly and safely.

The holder can be designed advantageously such that, when adding a throughflow unit, an operative connection of the transducer to the transducer connecting element and/or the transducer interface to the throughflow device transducer is automatically created. In an advantageous manner, this produces an efficient coupling of the throughflow unit to the blood testing device. Alternatively or additionally, the holder can be designed such that, while adding a throughflow unit, an operative connection of the transducer interface is automatically produced to the throughflow device transducer by means of which, advantageously, data from a transducer, which is arranged on or in the throughflow unit, can be transferred to the blood testing device also.

In a particular embodiment, the holder has at least one fastening element to fasten a throughflow unit. This ensures a particularly reliable fastening of the throughflow unit to the blood testing device. Alternatively, the throughflow unit can have a fastening element which manufactures the mechanical connection with the holder. In another design, the holder has a fastening element which collaborates with a counter fastening element of the throughflow unit by means of which a particularly stable attachment is produced.

In an advantageous embodiment, the blood testing device is designed to record at least one blood parameter. For this, the blood testing device has a signal and data processing unit which processes the signals received from the transducer, such as the sound reflected from blood cells, as well as determining corresponding blood parameters from them and passing them on to the output on the display device.

In particular, the blood testing device is designed to record at least the following blood parameters: oxygen saturation of the blood, CO₂ content of the blood, temperature of the blood, blood pressure, blood flow rate over time, blood speed and haemoglobin proportion in the blood. As a result, many blood parameters, relevant for the vital functions of the patient, can be monitored, in particular permanently, in an advantageous manner.

The throughflow unit according to the invention is designed and intended to be incorporated in an extracorporeal blood circuit so that blood flows along a flow direction through the throughflow unit and is suitable to be directly connected to a blood testing device according to the invention. The throughflow unit has at least one transducer connecting element and at least one throughflow device transducer.

By means of the throughflow unit, firstly therefore a blood testing device can be connected particularly easily mechanically and secondly a coupling to transfer energy and data can be made particularly easily both from the throughflow unit to the blood testing device as well as from the blood testing device to the throughflow unit.

In a special embodiment, the throughflow device transducer has a pressure transducer to record the pressure of the blood in the extracorporeal blood circuit.

In a further embodiment, the throughflow device transducer has a flow transducer. Thus, in an advantageous manner, this provides that the flow rate of the blood through the extracorporeal blood circuit per unit of time can be determined.

In another embodiment, the throughflow device transducer has an optical transducer. An embodiment of this type has the advantage that, for example, the oxygen saturation, the CO₂ content or the haemoglobin content of the blood can be determined.

Advantageously, the throughflow unit may also have several integrated transducers, in particular several of the transducers mentioned above.

Alternatively or additionally, the transducer attachment element can be designed for connecting to a throughflow device transducer of the blood testing device wherein the throughflow device transducer has a pressure transducer or a flow transducer or an optical transducer. As a result, different transducers of a blood testing device can be connected in different combinations to the throughflow unit in an advantageous manner.

In a special embodiment, the throughflow device transducer can have at least one light source to illuminate the blood flowing through the throughflow unit. Using this method, a specific amount of light can enter the blood by means of the transducer and detection light, due to the impact of the light (e.g. reflected light, scattered light or fluorescent light) leaving the blood, is detected by means of a light meter of the transducer and if necessary analysed, particularly with regard to the light intensity and/or the wavelength.

Alternatively or additionally, provision can be made advantageously that the throughflow device transducer has at least one sound source to impact the blood flowing through a throughflow unit with sound. This can be accomplished by directing a particularly adjustable sound into the blood by means of the transducer. Also, an acoustic sensor can also be provided which is reflected by the blood or receives scattered sound. A transducer of this type can be an ultrasound transducer in particular which operates on the basis of ultrasound and functions as a flow meter.

An embodiment is particularly advantageous in which the transducer is both a light source as well as a sound source. As a result, several, in particular, different blood parameters can be recorded simultaneously.

In particular, it can be advantageously provided that the transducer attachment element has a window. A sight contact can be seen through the window, for instance before the connecting of a blood testing device to the throughflow unit to which the blood is flowing through the throughflow unit. Thus, in an advantageous manner, medical personnel can see, for example, colour changes in the blood which indicate a change in the haemoglobin content in the blood.

In addition, an efficient connection of an optical transducer is possible since light from a light source of the optical transducer and/or light to be received from a sensor of the optical transducer can be propagated through the window.

Alternatively or additionally, the transducer attachment element of the throughflow unit can have a flexible membrane. The flexible membrane enables a movement contact to be made to the blood flowing through the throughflow unit.

The mechanical flexibility of the membrane makes possible, for example, the fast and uncomplicated coupling of a transducer designed as a pressure transducer of the blood testing device according to the invention. By means of this type of membrane, for example, an increase in the pressure of the blood in a throughflow unit or in an extracorporeal blood circuit, which causes the membrane to dome outwards, can be transmitted directly to the pressure transducer.

In a quite particularly advantageous and particularly compactly designed embodiment, both the transducer coupling element as well as the throughflow device transducer (and possibly other elements of this type) tangentially surround the longitudinal axis of the throughflow unit and/or are arranged axially apart relative to one another. In particular, they can be arranged in a common plane perpendicular to the direction of flow. For this to happen, the throughflow unit is short and compact in design in an advantageous manner with respect to its longitudinal extension. Thus the device can be used also in an area of bends in tubes or does not prevent the tube to be run flexibly. Usually, transducers are arranged longitudinally in the flow direction or at different positions.

In a further embodiment, the throughflow unit has plug-in connections for fluidic connections with tubes or sections of tubing of an extracorporeal blood circuit. The tubes can be plugged into the plug connections so that they are, in particular, non-slip and interlocking.

In particular, this guarantees that the interface between the tube and the throughflow unit is leakproof so that no blood can leak out of the extracorporeal blood circuit.

In particular, the plug-in connections can each have a disc. Each of the discs can function as a stop element for the respective tube which has to be attached. Alternatively or additionally, the discs can be arranged such that they mechanically guide the throughflow unit and a blood testing device during a connecting process relative to one another, wherein the blood testing device can be guided between the discs to the part of the throughflow unit, in particular so that they interlock.

In a particularly advantageous embodiment, the throughflow unit is produced from a flexible material, in particular a plastic. This reduces the risk of a break or the disturbance of the throughflow unit caused by a mechanical impact since, due to its flexibility, up to a certain degree it can change its shape without damaging its structure. In particular, the throughflow unit can have a flexible transparent tube or be designed in the form of a flexible transparent tube including its technical devices wherein the blood flows through the tube. The transparent tube, besides the already stated advantage regarding the flexibility, also provides, in an advantageous manner, a direct observation of the blood. The blood testing device can also be designed advantageously so that it can be operatively connected to a simple, in particular transparent, piece of tubing as a throughflow unit (without an incorporated transducer) in order to record blood parameters.

Alternatively, the throughflow unit according to the invention can be designed as a cuvette made from polycarbonate or MABS.

Of particular advantage is a system which has a throughflow unit according to the invention and a blood testing device according to the invention. This allows medical personnel to quickly and simply measure and/or monitor blood parameters in an extracorporeal blood circuit of a patient who is connected, in particular, to a heart/lung machine.

In the drawing, the subject matter of the invention is illustrated in an exemplary and schematic fashion and is described using the figures in the following text wherein identical and identically-acting elements are also mostly provided in different embodiments with the same reference labels. They show in:

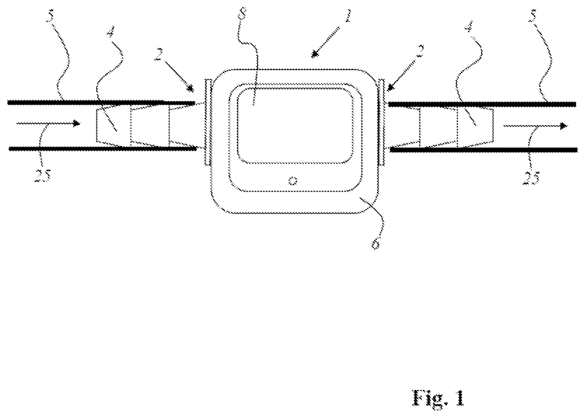

FIG. 1 a top view of a first embodiment of a system according to the invention, comprising a first embodiment of a blood testing device and a first embodiment of a throughflow unit,

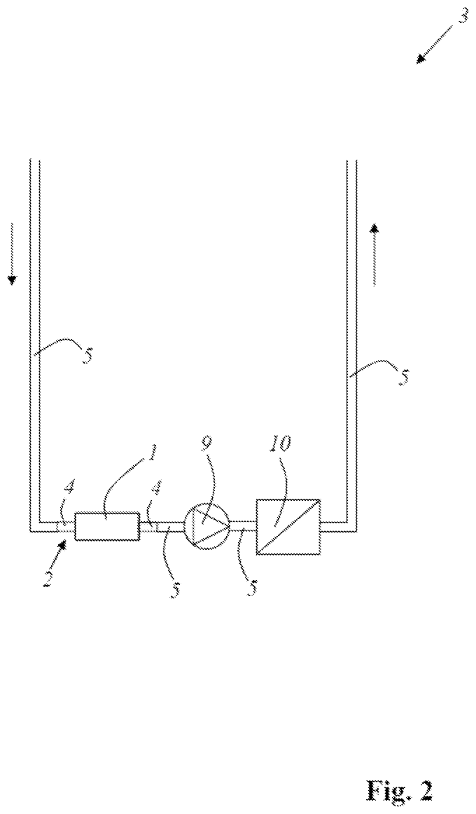

FIG. 2 an extracorporeal blood circuit with the system according to the invention,

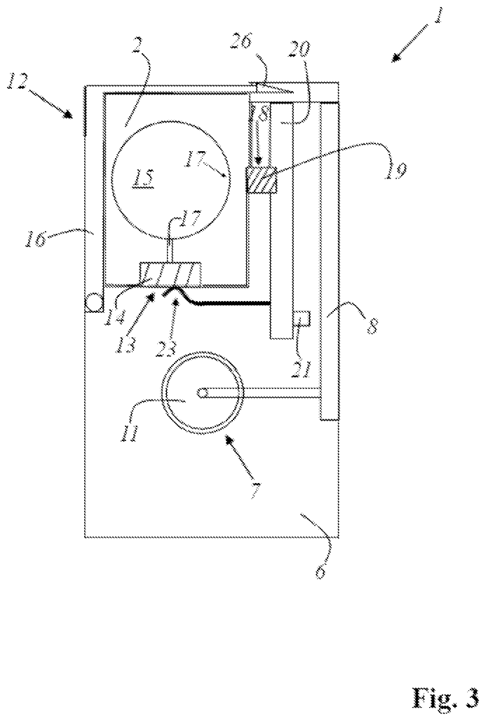

FIG. 3 a sectional view of a second embodiment of a system according to the invention, comprising a second embodiment of a blood testing device according to the invention and a second embodiment of a throughflow unit according to the invention,

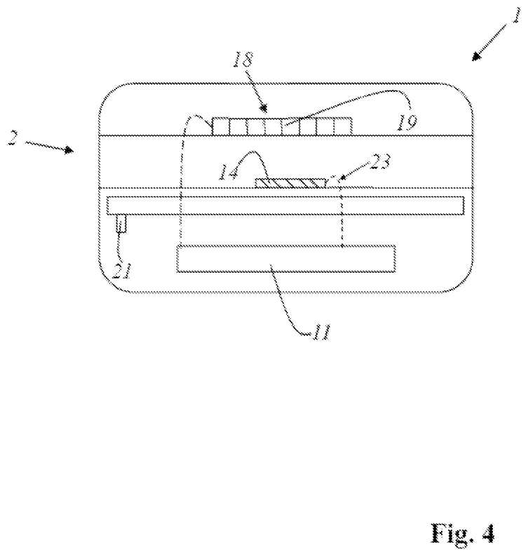

FIG. 4 a sectional view of a third embodiment of a system according to the invention, comprising a third embodiment of a blood testing device according to the invention and a third embodiment of a throughflow unit according to the invention,

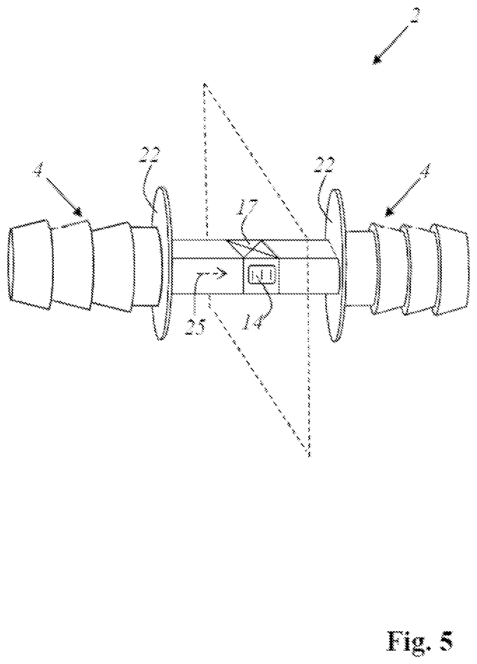

FIG. 5 a perspective view of a fourth embodiment of a throughflow unit according to the invention, and

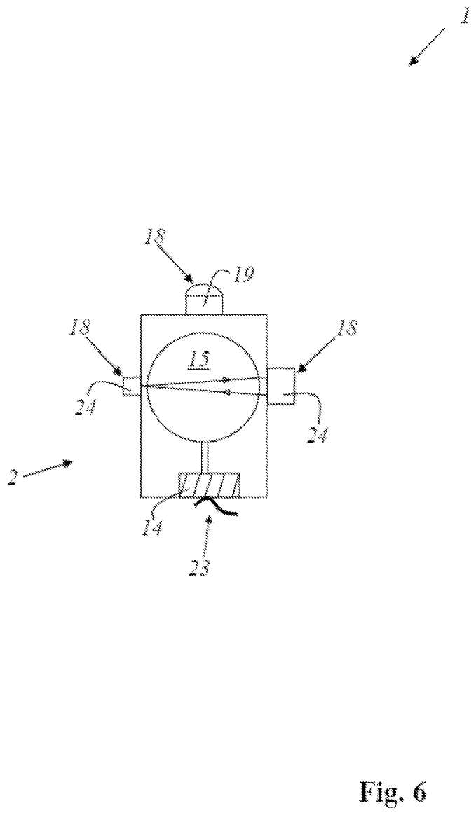

FIG. 6 a sectional view of a fifth embodiment of a throughflow unit according to the invention.

FIGS. 1 and 2 show a top view of an embodiment of a system according to the invention comprising the blood testing device 1 and a throughflow unit 2. The blood testing device 1 is connected to a throughflow unit 2 of an extracorporeal blood circuit 3. The blood testing device 1 is designed to record blood parameters of the blood flowing through the throughflow unit 2.

The throughflow unit 2 has two plug connections 4 which are connected with tubes 5 of an extracorporeal blood circuit 3. The direction of flow 25 is indicated by arrows in FIGS. 1 and 2.

The blood testing device 1 has a housing 6, in or on which an (not illustrated in FIGS. 1 and 2) electrical energy storage device 7 and an indicator device to indicate 8 the recorded blood parameters are arranged.

The blood testing device 1 of the system can be used quickly and without having to be connected to a power circuit or to other devices. The blood testing device 1 is operated without cables so that, on the one hand, it is particularly easy to manage and, on the other hand, the personnel treating the patient are not inconvenienced by cables hanging about, or detaching the cables is prevented by people moving around. Furthermore, the blood testing device 1 is particularly compact in design as a manually operated device.

FIG. 2 shows an extracorporeal blood circuit 3 with the throughflow unit 2, to which the blood testing device 1 is connected. The extracorporeal blood circuit 3 also has a pump 9 for pumping blood and an oxygenator 10. The devices are connected by tubes 5 wherein the connection to the blood circuit of the patient is also done by means of tubes 5.

FIG. 3 shows a second embodiment of a system according to the invention, comprising a second embodiment of a blood testing device and a second embodiment of a throughflow unit in a sectional drawing. The blood testing device 1 is designed autonomously and can be operated without a cable connection to other devices of a heart/lung machine or to an external energy source. The blood testing device 1 has an electrical energy storage unit 7 which, in the embodiment shown, is a battery 11 which supplies the remaining components of the blood testing device 1 with electrical energy.

The blood testing device 1 has a holder 12 into which the throughflow unit 2 is fixed. The holder 12 has a flap 16 rotatably pivoted on the housing 6 which opens to insert the throughflow unit 2 and can be closed again after the insertion of the throughflow unit 2 so that the throughflow unit 2 makes an interlocking connection to the blood testing device 1. The upper part of the flap 16 is flexible and has a snap-in lug 26 at its end.

The throughflow unit 2 is attached to the holder 12 non-destructively and without requiring tools. The holder 12 enables a fast and efficient coupling and uncoupling of the throughflow unit 2 to be made. Also, an immediately recognisable securing ability is created for a user by the holder 12, by means of which the risk of faulty operation by incorrect attachment is reduced. In particular, this guarantees that the blood testing device 1 and the throughflow unit 2 are connected together correctly and safely.

The throughflow unit 2 has a transducer attachment element 17 and the blood testing device 1 has a transducer 18. The transducer 18 is an optical transducer 19. The transducer attachment element 17 is formed by the transparent wall of the throughflow unit 2 through which the light to record an optical measurement signal can penetrate.

When the throughflow unit 2 is added, an operative connection of the transducer 18 of the blood testing device 1 to the transducer attachment element 17 of the throughflow unit 2 is automatically created and an operative connection of the transducer interface 23 to the throughflow device transducer 13 of the throughflow unit 2 is automatically produced. By doing so, advantageously an effective coupling 1 of the throughflow unit 2 to the blood testing device is produced. Advantageously, data can also be transferred from the transducer 13 to the blood testing device 1 via the transducer interface 23.

The throughflow unit 2 has a throughflow device transducer 13 which is a pressure transducer 14 according to the embodiment illustrated in FIG. 3. The pressure transducer 14 is connected by means of a transducer coupling element 17 with the interior 15 of the throughflow unit 2 through which the blood flows, so that the pressure of the blood occurring in the inner space is transferred to the pressure transducer 14. The transducer attachment element 17 has a membrane by means of which the pressure of the blood is transferred to the pressure transducer 14.

Alternatively, the transducer attachment element 17 can also be designed, for example, by a compression die which acts on the pressure transducer 14.

The blood testing device 1 also has an electronic device 20, like, for example, a programmable logic controller (PCL), and an indication device for the indication 8 of the recorded blood parameters.

Furthermore, the blood testing device 1 has a transmission and receiving device 21 for transmitting and receiving measurement signals wherein the transmission and receiving device 21 in this embodiment is designed for wireless transmission.

FIG. 4 shows a sectional view of a third embodiment of a blood testing device 1 according to the invention. The blood testing device 1 has an optical transducer 19 as a transducer 18 which is arranged opposite the pressure transducer 14 of the throughflow unit 2.

The battery 11 of the blood testing device 1 supplies electrical power to both the optical transducer 19 of the blood testing device 1 as well as the pressure transducer 14 of the throughflow unit 2. The battery 11 is connected by a cable to the transducer 19 and to the pressure transducer 14 by means of the schematically depicted transducer interface 23.

FIG. 5 shows a perspective view of a fourth embodiment of a throughflow unit 2 according to the invention.

The throughflow unit 2 has two plug-in connections 4 to connect fluidically with (not shown in this figure) tubes 5 of an (not shown in this figure) extracorporeal blood circuit 3. The plug-in connections 4 each have a disc 22. The discs 22 function as a stop for the tubes 5 which are to be connected. The discs 22 fulfil a double function simultaneously, that is, to act as guides for a blood testing device 1 to be connected to the throughflow unit 2 as soon as it is plugged to the throughflow unit 2 to make the connection. Furthermore, the discs 22 are arranged such that they are able to make an interlocking connection of the blood testing device 1.

The discs are also arranged such that they undertake the guidance of a blood testing device 1 to attach to the throughflow unit 2 wherein the blood testing device 1 is guided between the discs 5 on to which the intermediate part of the throughflow unit 2 is interlockingly pushed.

The throughflow unit 2 also has a pressure transducer 14 and a transducer attachment element 17 for the automatic connection of a transducer 18 (not shown in this figure) of a blood testing device 1. The transducer attachment element 17 and the pressure transducer 14 are arranged on one common plane (shown schematically as a dotted line) perpendicular to the direction of flow 25. By doing so, because of its longitudinal extension, the throughflow unit 2 is designed to be short and therefore compact.

FIG. 6 shows a sectional view of a fifth embodiment of a throughflow unit 2 according to the invention.

The throughflow unit 2 is defined as a blood testing device 1 according to the invention wherein, for simplification, only the transducers 18 of the blood testing device 1 are shown. The blood testing device 1 has a transducer 18 which, as an ultrasonic transducer 24, measures the blood flow per unit of time. The ultrasonic transducer 24 has an ultrasonic transmitter and a reflector to reflect the ultrasonic waves. Also, the blood testing device 1 (not fully illustrated) has an optical transducer 19 to determine the oxygen content and/or CO₂ content in the blood.

Since several transducers 18 of different types are present, different technical possibilities, also in combination, can be effected.

### REFERENCE LIST

1 Blood testing device

2 Throughflow unit

3 Blood circuit

4 Plug-in connection

5 Tube

6 Housing

7 Energy storage

8 Display device for displays

9 Pump

10 Oxygenator

11 Battery

12 Holder

13 Throughflow device transducer

14 Pressure transducer

15 Inner space

16 Flap

17 Transducer connecting element

18 Transducer

19 Optical transducer

20 Programmable logic controller

21 Transmission and receiving device

22 Disc

23 Transducer interface

24 Ultrasonic transducer

25 Flow direction

26 Snap-in lug

1.-14. (canceled)

15. A blood testing device configured for direct connection to a throughflow unit for blood, in particular a throughflow unit of an extracorporeal blood circuit, and for the recording of blood parameters of the blood flowing through the throughflow unit, characterized in that the blood testing device can be operated without cables.

16. A blood testing device according to claim 15, characterized in that the blood testing device has a housing, in or on which an electrical energy storage device and an indicator device for displaying the recorded blood parameters are arranged.

17. A blood testing device according to claim 15, characterized in that it is autonomous.

18. A blood testing device according to claim 15, characterized by at least one transducer.

19. A Blood testing device according to claim 18, characterized in that the transducer
a. has at least one light source for illuminating the blood flowing through a throughflow unit with light and/or
b. a sound source for enveloping the blood flowing through a throughflow unit with sound and/or
c. has a pressure transducer and/or
d. has a flow transducer and/or
e. has an optical transducer.

20. A blood testing device according to claim 15, characterized by at least a transducer interface.

21. A blood testing device according to claim 20, characterized in that the transducer interface to designed to,
a. transfer energy from the electrical energy storage unit to a throughflow device incorporated in a throughflow unit and/or
b. relay measurement signals from a throughflow device transducer incorporated in a throughflow unit.

22. A blood testing device according to claim 20, characterized in that the transducer interface is designed for connecting to a throughflow device transducer which has a pressure transducer or a flow transducer or an optical transducer.

23. A blood testing device according to claim 15, characterized by a holder which can be fixed in or on the one throughflow unit.

24. A blood testing device in particular according to claim 1, characterized in that the blood testing device is designed to record at least one of the following blood parameters:
a. Oxygen saturation of the blood,
b. CO₂ content of the blood
c. Temperature of the blood,
d. Pressure of the blood,
e. Flow quantity of the blood per unit of time,
f. Flow speed of the blood,
g. Haemoglobin proportion in the blood.

25. A throughflow unit, which is configured to be introduced into an extracorporeal blood circuit such that blood flows along a flow direction through the throughflow unit and is designed to be coupled directly to a blood testing device according to claim 15, characterized by at least one transducer attachment element and at least one throughflow device transducer.

26. A throughflow unit according to claim 25, characterized in that
a. the throughflow device transducer has a pressure transducer or a flow transducer or an optical transducer, and/or that
b. the throughflow device transducer has at least one light source and/or a sound source to flood the blood flowing through the throughflow unit with light and/or sound and/or that
c. the transducer attachment element is designed for connecting to a transducer of a blood testing device which has a pressure transducer or a flow transducer or an optical transducer, and/or that
d. the transducer attachment element has a window or a flexible membrane.

27. A throughflow unit according to claim 25, characterized in that both the transducer attachment element as well as the throughflow device transducer run tangentially around the longitudinal axis of the throughflow unit and/or are arranged relative to one another axially spaced apart.

28. A system having a throughflow unit according to claim 25 and the blood testing device.
